(19) United States
(12) Patent Application Publication
(10) Pub. No.: US 2006/0165662 A1
(43) Pub. Date: Jul. 27, 2006

(54) Isolation of target cells, capillaries and microorgans

(76) Inventors: Minoru Takemoto, Goteborg (SE); Christer Betsholtz, Goteborg (SE)

Correspondence Address: ALBIHNS STOCKHOLM AB, BOX 5581, LINNEGATAN 2, SE-114 85 STOCKHOLM; SWEDENn, STOCKHOLM, SE

(21) Appl. No.: 10/513,027

(22) PCT Filed: Apr. 30, 2003
(86) PCT No.: PCT/SE03/00672

§ 371 Date: Jul. 18, 2005

Related U.S. Application Data
(60) Provisional application No. 60/376,840, filed on May 2, 2002.

(51) Int. Cl. A61K 35/12; C12Q 1/68; C12N 1/08; C12N 5/08

(52) U.S. Cl. 424/93.7; 435/6; 435/270; 530/412; 435/366

(57) ABSTRACT

An isolation method for target cells, capillaries and microorgans comprises perfusion of an organism with a solution containing magnetic beads, removal of a selected tissue or region of the organism, digestion of the portion removed and isolation of the target cells, capillaries or microorgans using a magnet. Filtration may be used to facilitate the method.

### FIELD OF THE INVENTION

The present invention relates generally to methods of isolating regions of interest from an organism. More specifically, the invention relates to methods of isolating specific cells, capillaries or microorgans from an organism.

### BACKGROUND OF THE INVENTION

As genome projects^{1,2} near completion and begin to reveal a wealth of new information, researchers are now worling toward a better understanding of which region of the genome corresponds to specific cellular functions and states of differentiation. This new research often requires the isolation of a highly homogenous population of cells and/or microorgans from in vivo situations. Some methods are available for certain species or target cell types, however, improved isolation methods are still needed in the art.

For example, one microorgan of interest is the kidney glomerulus. The kidney glomerulus is a tuft of capillaries composed of many loops surrounded by epithelial cells (called podocytes) and held together by a core of mesangial cells and matrix. The main function of the glomerulus is to constitute a permeable, size-selective barrier across which blood is filtered to produce primary urine. The three highly specialized cell types of the glomeruli; fenestrated endothelial cells, mesangial cells and podocytes, together with the glomerular basement membrane constitute approximately 10% of the whole kidney tissues. Although endothelial cells and pericytes exist outside the glomerulus, their phenotype within the glomerulus is quite distinct from related cells elsewhere⁷.

To allow efficient selective filtration, cellular and matrix components of the glomerulus are endowed with specialized features. Developments using knock-out and trnnsgenic analyses in mice have been instrumental in identifying molecules important for kidney development, especially in the early stages of nephron or glomerulus development³⁻⁵. Gene products known to play essential roles in glomerulus finction and pathology⁶ have also been described. Despite this, knowledge remains limited with regard to late stage glomerulogene1is^{7,13,14}, steady-state function of the mature glomerulus, and specialized features pf the individual glomerulus cells.

Specific limitations hampering advances in the field include the low abundance of glomerulus cells and difficulties in harvesting glomerulus cells from cell culture due to their inability to retain differentiated features in culture. Methods to isolate glomeruli from rat¹⁵ and rabbit¹⁶ using sieving techniques have been described. These methods have not proven entirely practical for isolation of pure murine glomeruli since sieving cannot always distinguish between glomeruli and tubules, both having a similar diameter. Isolation of murine glomeruli after Fe₃O₄ perfusion has been reported¹⁷, however, the lack of isolation efficiency of this method makes it undesirable. Since mice are widely used as experimental models of development and disease, it would be of great benefit to have improved methods for murine glomerulus isolation. Isolation methods that can be applied to other experimental models, for example monkeys, as well as to humans, are especially desirable.

In addition to the need felt by researchers investigating kidney glomeruli, the absence of efficient microorgan or target cell isolation methods plagues researchers in other areas. It would benefit researchers studying cerebrospinal fluid and conducting brain or spinal cord research to have a better method to isolate the miute capillaries throughout the pia mater. Applicability of that improved isolation method to other blood vessel networks would make it even more preferable. It would benefit researchers in the field of pancreatic structure and function and diabetes research to have a new, efficient method to isolate islets of Langerhans. Current isolation techniques such as density gradient, immunological recognition, or sonication, for example the method described in U.S. Pat. No. 5,879,939, should be improved upon to better isolate those 1 or 2% of pancreas cells that form the islets of Langerhans. It would benefit researchers investigating vision dysfunction and vasculogenesis to have a better isolation method for the vasculature of the choriod. Thus, there has remained a substantial need for new and better methods of isolating microorgans and/or target cells.

### SUMMARY OF THE INVENTION

The present invention provides a new method for the isolation of target cells, capillaries or microorgans. The novel method improves upon traditional isolation methods due to the multiple applications of the technique, the simplicity and efficiency of the method, and the specificity and purity of results obtained thereby.

According to a first embodiment of the present invention, a method of isolating target cells is provided, comprising perfusing an organism with a solution containing magnetic beads, removing a selected tissue or region containing target cells from the organism, digesting the selected tissue or region to separate target cells from associated cell types, and magnetically isolating target cells from the digested selected tissue or region, wherein the diameter of the magnetic beads is approximately equivalent to the capillary diameter of the region comprising the target cells. Optionally, the digesting can be performed using collagenase. The method can further comprise filtering the digested selected tissue or region prior to the magnetic isolation step. Optionally, a 100 μm filter may be used. The invention further comprises isolated target cells obtained thereby. Optionally, the target cell may be a pericyte, vascular smooth muscle cell, or astrocyte.

According to a further embodiment of the present invention, a method of isolating target capillaries is provided, comprising perfusing an organism with a solution containing magnetic beads, removing a selected tissue or region containing target capillaries from the organism, digesting the selected tissue or region to separate target capillaries from associated cell types, and magnetically isolating target capillaries from the digested selected tissue or region, wherein the diameter of the magnetic beads is approximately equivalent to the diameter of the target capillaries. Optionally, the digesting can be performed using collagenase. The method can flrter comprise filtering the digested selected tissue or region prior to the magnetic isolation step. Optionally, a 100 μm filter may be used. The invention frther comprises isolated capillaries obtained thereby. Optionally, the target capillary may be a dermal capillary, retinal capillary, brain capillary, perineural plexus capillary, skeletal muscle capillary, tumor capillary or heart capillary.

According to a further embodiment of the present invention, a method of isolating a microorgan is provided, comprising perfusing an organism with a solution containing magnetic beads, removing a selected tissue or region containing the microorgan from the organism, digesting the selected tissue or region to separate the microorgan from associated cell types, and magnetically isolating the microorgan from the digested selected tissue or region, wherein the diameter of the magnetic beads is approximately equivalent to the capillary diameter of the microorgan Optionally, the digesting can be performed using collagenase. The method can further comprise filtering the digested selected tissue or region prior to the magnetic isolation step. Optionally, a 100 μm filter may be used. The isolated microorgan might be kidney glomeruli, islets of Langerhans, or endocrine glands, among others.

According to a furher embodiment of the present invention, a method of isolating a microorgan is provided, comprising perring an organism with a solution containing magnetic beads, perfusing an organism with a digestion solution, removing a selected digested tissue or region containing the microorgan from the organism, and magnetically isolating the microorgan from the digested selected tissue or region, wherein the diameter of the magnetic beads is approximately equivalent to the capillary diameter of the microorgan. Optionally, the digestion solution can be collagenase. The microorgan may be islets of Langerhans.

The organism used to practice the present invention may be a mammal, for example, a mouse, rat, rabbit, guinea pig, cat, dog, pig, cow, monkey, or human. The organism may be at any stage in development, for example, embryonic, neonatal, juvenile, or adult. The invention further comprises isolated microorgans obtained thereby. Isolated microorgans and/or target cells may be used as tissues for transplantation.

According to a furher embodiment of the present invention, a method of obtaining genetic material is provided, comprising obtaining at least one target cell, capillary or microorgan according to the present invention, and removing or isolating the genetic material from the at least one target cell, capillary or microorgan.

According to a further embodiment of the present invention, a method of obtaining proteins is provided, comprising obtaining at least one target cell, capillary or microorgan according to the present invention, and removing or isolating the genetic material from the at least one target cell, capillary or microorgan.

### BRIEF DESCRIPTION OF THE DRAWINGS

FIG. 1 is a diagrammatic representation of the five principal steps of an isolation. technique according to the present invention; involving: Step 1. Perfusion with Dynabeads diluted in PBS through the heart, Step 2. Removing Kidneys and Mincing into 1 mm³ pieces, Step 3. Digestion of tissue with Collagenase, Step 4. Filtration of the tissue with 100 μm cell strainer, Step 5. Isolation of glomeruli by a magnet.

FIG. 2A shows results of histological examination of adult murine kidney following magnetic bead perfusion and staining with hematoxylin and eosin, where the bar represents 50 μm;

FIG. 2B shows a magnified view of FIG. 2A, where the bar represents 50 μm and where arrows point to magnetic beads (not all beads are marked with arrows);

FIG. 3A shows results of histological examination of mice glomeruli following magnetic bead perfusion where the bar represents 50 μm;

FIG. 3B shows a magnified view of FIG. 3A where the bar represents 50 μm;

FIG. 3C shows a further magnified view of FIG. 3A where the bar represents 50 μm and where arrows point to magnetic beads (not all beads are marked with arrows);

FIG. 4A shows an electromicrograph of a whole isolated adult mice glomerulus, where the bar represents 10 μm;

FIG. 4B shows a magnified electromicrograph of the glomerulus of FIG. 4A showing the interdigitating pericyte foot process, where the bar represents 10 μm;

FIG. 4C shows a magnified electromicrograph of the glomerulus of FIG. 4A showing the constant width of the filtration slit, where the bar represents 100 nm;

FIG. 5A shows a transmission electron microscope view of an isolated adult mouse glomerulus where Cl indicates capillary lumen, En indicates Endothelial cell, MC indicates Mesangial cell, and Po indicates Podocyte, the arrow indicates fenestrae of endothelium, arrowheads indicate podocyte slit diaphragms. and. the bar represents 2.5 μm;

FIG. 5B shows a magnified transmission electron microscope view of the glomerulus of FIG. 5A showing the filtration barrier and part of the fenestrated endothelium, where the asterisk indicates an apparently unaltered basal lamina, arrowheads indicate filtration slits with diaphragms, and the bar represents 100 nm;

FIG. 6 shows yield data for glomeruli per adult mouse isolated according to the present invention where the bar indicates the mean value;

FIG. 7 shows purity data for glomeruli isolated according to the present invention where the bar indicates the mean value;

FIG. 8 shows total RNA in glomeruli isolated according to the present invention where the bar indicates the mean value;

FIG. 9 shows electrophoretic ethidium bromide staining of isolated RNA where lanes 1 and 2 contain 8 μm of total RNA from glomeruli and lanes 3 and 4 contain 10 μm total RNA;

FIG. 10 shows results of a northern blot analysis of RNA from glomeruli where a Nephrin cDNA probe was used as a podocyte marker, a Tie 2 cDNA probe was used as a capillary endothelial cell marker, and Beta-actin and GADPH cDNA probes were used to assess expression of house keeping genes;

FIG. 11A shows histological results after hematoxylin and eosin staining of a vesicle stage glomerulus, where the bar represents 50 μm;

FIG. 11B shows histological results after hematoxylin and eosin staining of a S-shaped body stage glomerulus, where arrows point to magnetic beads (not all beads are marked with arrows) and the bar represents 50 μm;

FIG. 11C shows histological results after hematoxylin and eosin staining of a capillary loop stage glomerulus, where arrows point to magnetic beads (not all beads are marked with arrows) and the bar represents 50 μm;

FIG. 11D shows histological results after hematoxylin and eosin staining of a maturing stage glomerulus, where arrows point to-magnetic beads (not all beads are marked with arrows) and thebar represents 50 μm; and

FIG. 12 shows isolated glomeruli of variable size and at different developmental stages.

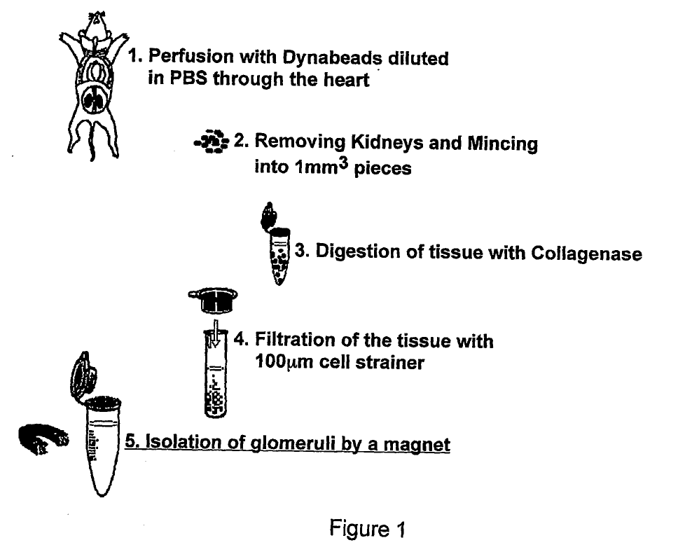

### DETAILED DESCRIPTION

An efficient method for the isolation of select cells, capillaries and/or microorgans has been developed. The method is advantageous both for morphological studies and for keeping intact the mRNA and protein profiles. The preservation of mRNA flows from the relative speed with which the method can be performed and the initiation of the method in an intact organism. One specific use of the method described is the isolation of developing or adult murine glomeruli. The method can produce large-scale isolation of intact glomeruli. The method translates well to other species, such as rabbits. Other applications include isolation and recovery of other microorgans and capillaries from various species of interest.

The inventive method uses magnetic beads to isolate the cells, capillary or microorgan of interest. In the following experiments, spherical DYNABEADS® (Dynal) were used, however, other equivalent products are commercially available and could be substituted. DYNABEADS® contain iron, providing magnetic properties within a magnetic field¹⁸. The bead surface is smooth with a coated monodisperse polymer shell that reduces direct damage to tissues when beads are perfused. The shell also prevents toxic exposure to iron. The diameter of bead chosen necessarily varies depending on the application. The diameter chosen corresponds to the diameter of the capillary that will be selectively embolized with magnetic beads, facilitating isolation with a magnet. For murine glomeruli isolation, 4.5 μm diameter beads are the appropriate size to specifically embolize the glomerular capillaries and to minimize cell damage.

The elastic nature of capillaries must be considered when selecting bead sizes. Beads of the chosen diameter will be able to travel through the circulatory system of the perfused organism until they reach the target. The beads can proceed some distance into the capillary or region of interest due to circulatory pressure and elastic expansion of the capillary diameter. As the beads slow to a near standstill in the selected region, the elasticity will no longer permit passage of the beads and the capillary will become embolized by the beads.

Islets of Langerhans are a microorgan that can be isolated according to the present inventive method. The microcirculation system within the islets can be filled with appropriately sized beads and quickly isolated from an organism of interest.

The present invention also is well suited to isolation of the capillary network in the choriod, the vascular network between the retina and sclera. This can provide a source of cells for analysis and experimentation in various fields including vision disorders and vasculogenesis.

Similarly, the choroids plexus, vascular network of minute capillaries throughout the pia mater, can be isolated to further research in various fields of brain, spinal cord, and cerebral spinal. fluid research. The present isolation method is particularly advantageous for isolation brain capillaries because they are among the smallest in an organism, and therefore susceptible to very specific embolization based on bead diameter.

In some instances a specific manufacturer or supplier is noted for reagents or equipment, however, materials necessary for the method typically can be obtained from a number of sources known to one of skill in the art.

### EXAMPLE I

### Isolation of Glomeruli

Mice, either C57B16 mice or 129/sv mice, or hybrids of those two species, were anesthetized by intraperitoneal injection of 17 μl/g of Avertin (2,2,2-tribromoethyl and tertiary amyl alcohol). 8×10⁷ DYNABEADS® M-450 Tosylactivated (4.5 μm diameter) (Dynal, Oslo, Norway) were inactivated according to the manufacturer's instructions and diluted in 40ml of either phosphate buffered saline (PBS) or HBSS (Hanks Buffered Saline Solution). The anesthetized mice were perfused with the magnetic bead solution through a hole excised in the right atria of the heart.

Following perfusion, the mice were sacrificed, kidneys were removed and minced into 1 mm³ pieces. The minced kidneys from newborn mice were digested in collagenase (1 mg/ml Collagenase A (Roche Diagnostic), 100 U/ml Deoxyribonuclease I (Invitrogen) in Hank's balanced salt solution (HBSS) (Invitrogen)) at 37° C. for 15 minutes with gentle agitation. The minced kidneys from adult mice were similarly digested in collagenase for 30 minutes. Collagenase is usefull for enzymatic isolation of cells because it dissolves collagen, a prominent structural protein within tissue. Alternative enzymes or digestion methods could have been used, however, collagenase is readily available and works relatively quickly.

The collagenasedigesled tissue was gently pressed through a 100 μm cell ser (Falcon) using a flattened pestle. The cell strainer was then washed with 5 ml of HBSS. The filtered cells were passed through a new 100 μm cell strainer without pressing, and the cell sainer was washed with 5 ml of HBSS. The cell suspension was.centrifuged at 200×g for 5 minutes. The supernatant was discarded and the cell pellet was resuspended in 2 ml of HBSS.

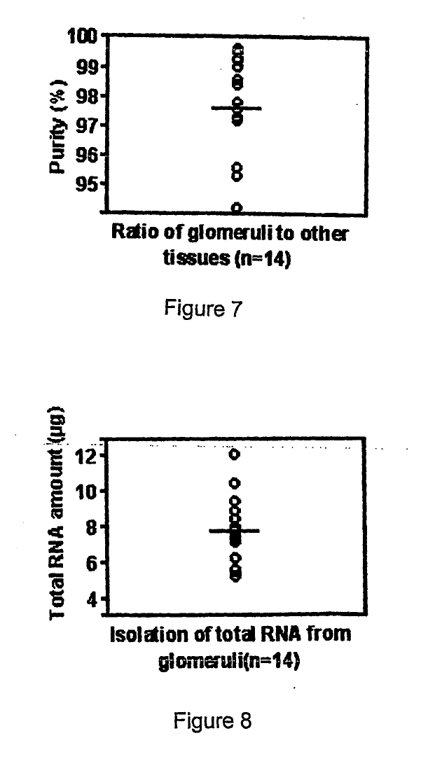

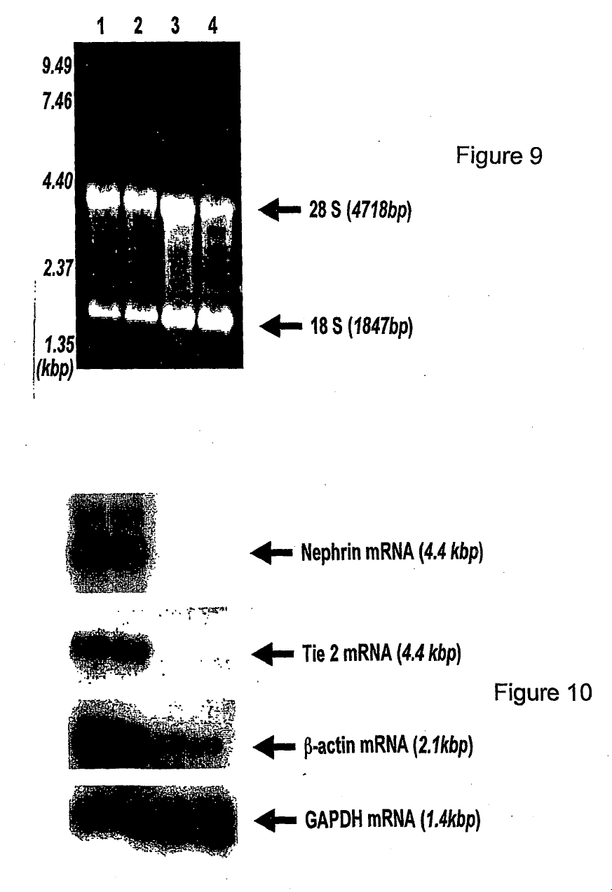

Glomeruli containing magnetic beads were gathered by a Magnetic Particle Concentrator (MPC, Dynal) and washed at least three times with HBSS. With the exception of the collagenase digestion step, tissues were kept at 4° C. The number of glomeruli retrieved from one adult mouse was 20131±4699 (mean±S.D., n=14) with a purity of 97.5±1.7% (FIGS. 6 and 7, where the bar indicates the mean value for results from 14 mice aged 3-24 weeks). This is consistent with the amount of glomeruli found in an adult mouse¹¹. This was also confirmed by the lack of glomeruli in the tissues that were not gathered by the MPC (data not shown).

The feasibility of the method to isolate the glomeruli of developing mice, as well as those of newborns and adults, is advantageous because it provides researchers with an efficient, reliable method of isolation of the immature structures. This method makes downstream research feasible, including transcript profiling and proteomic analysis of developing, healthy and diseased glomeruli. The inventive method makes possible the application of techniques for systematic analysis of gene and protein expression, such as EST-sequencing²¹, serial analysis of gene expression²², DNA microarray hybridization²³ and proteomics²⁴. The relative speed with which the method can be completed is important for preserving the in vivo transcript and protein profile.

### EXAMPLE II

### Morphology of Isolated Glomeruli

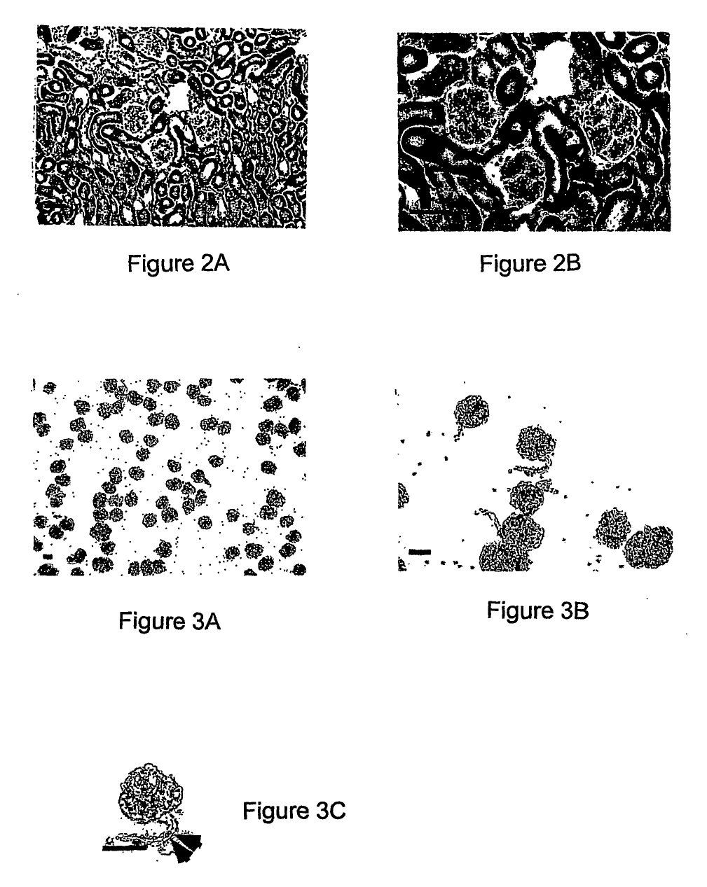

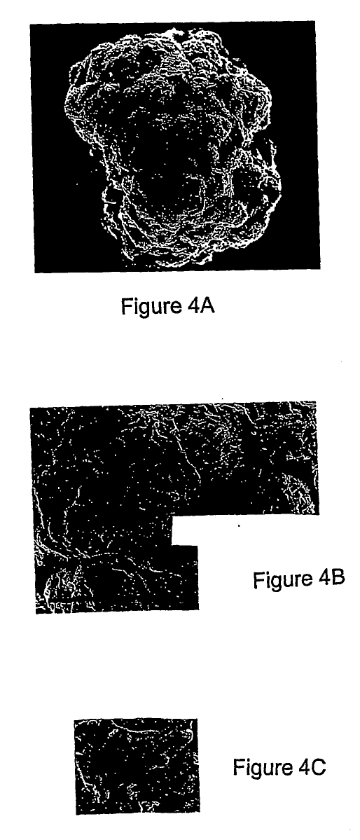

Murine kidneys perfused with magnetic beads were obtained according to Example I. The kidneys were snap frozen, sectioned, and stained with Hematoxylin-Eosin. A light microscope was used to examine the stained kidney sections. Hematoxylin and Eosin staining of the kidneys from mice perfused with magnetic beads revealed that the beads were mainly distributed in the glomernli and only a few beads could be seen in the surrounding renal tissues (results from a four week old mouse, FIGS. 2A and 2B). Collagenase digestion of the kidney had little effect on the glomerular structure. Magnetic beads accumulated in the glomeruli vessels, making the glomeruli easy to isolate using a magnet and providing a low degree of contamination with undesired tissues (rusults from an adult mouse, FIGS. 3A, 3B and 3C). Almost all isolated glomeruli were lacking the Bowman's capsule. Some of the isolated glomeruli still had a portion of the afferent arteriole and/or the efferent arteriole attached.

Scanning and transmission electron microscopes were also used. to observe stained kidney sections, following the methods described by Friedman P. L., et al., Braet F., et al.^{8,9}. The morphology of the isolated glomeruli thus observed is shown in FIG. 3. Most of the isolated glomeruli maintained good morphology. Minor detachment of podocytes could occasionally be seen. The isolated glomeruli were well covered by podocytes. The fine structures of the podocyte slit diaphragms and fenestrated endothelial cells could easily be seen using electron microscopes.

### EXAMPLE III

### RNA Integri of Isolated Glomeruli

Murine glomeruli were obtained according to Example I. Total RNA was isolated from the glomeruli using an RNase mini kit (Qiagen) according to manufacturer's instructions. Northern blot analysis was performed according to Scheidl, J. S., et al.¹⁰ using ³²Plabeled Nephrin ODNA (Dr. Heli Putaala, Karolinska Institute, Sweden) as a marker for podocytes and Tie-2 cDNA (Dr. Tom Sato, University of Texas Southwestern Medical Center, USA) as a marker for endothelial cells. Glyceraldehyde-3-phosphate dehydrogenase (GAPDH) and beta-actin cDNA probes were used to assess expression of housekeeping genes and therefore help determine the purity of the yield.

Nephrin is a recently identified protein expressed only at the glomerular podocyte slit diaphragm, it has been reported to be mutated in congenital nephrotic syndrome of the Finnish type¹⁹. Tie-2 is an endothelium-specific receptor tyrosine kinase, which binds to Angiopoietins 1 and 2²⁰. Its strong expression in the kidney glomerulus compared with whole kidney parallels known differences in proportion, the glomerulus consisting of approximately 50% endotheiial cells, whereas the rest of the kidney comprises far fewer endothelial cells.

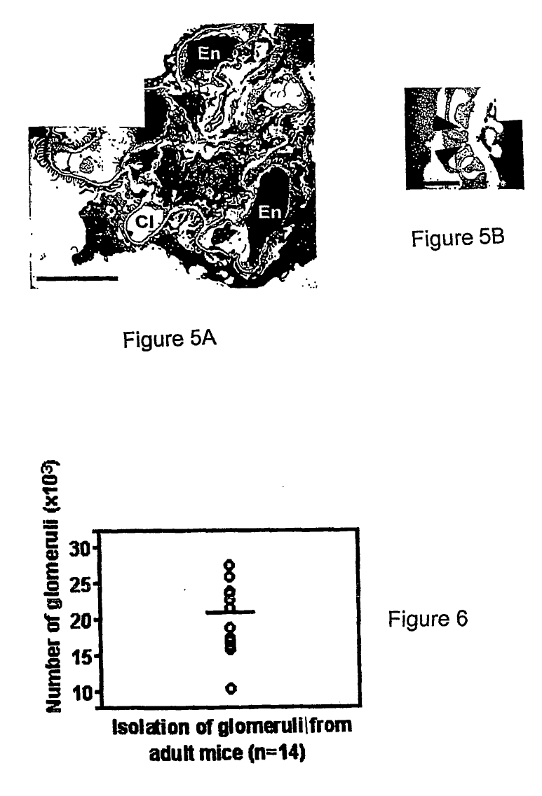

The amount of total RNA retrieved from isolated glomeruli of one adult mouse was estimated to be 7.9 μg (FIG. 8). There were no signs of RNA degradation during the isolation procedure, the integrity of the RNA was high. These results are depicted in FIG. 5, where lanes 1 and 2 contain 8 μg of total RNA from isolated glomeruli and lanes 3 and 4 contain 10 μg of total RNA from snap frozen whole kidney. This result is likely due to the simplicity and speed of the novel isolation method.

Northern blot analysis confirmed total RNA results and enrichment of glomeruli. Specifically, the analysis demonstrated that cDNA corresponding to podocyte or endothelial cell mRNA recognized abundant transcripts in glomerulus total RNA but not in whole kidney total RNA (FIG. 10). Ethidium bromide staining and hybridization against GAPDH and beta-actin probes verified the equal integrity and loading of the RNA samples.

This large collection of RNA and the applicability of the present method to other cells, capillaries, or microorgans of interest in varying species demonstrates the research potential from using the present method. Rapid, high yields of relatively pure genetic material will be of great use to researchers in a multitude of fields.

As shown in Examples II and III, beyond examination of mere morphology, the cells or organs collected by the present method tend to be excellent candidates for genetic analysis. Their good condition upon collection allows researchers to design study protocols that seek to explain the timing of gene expression and genetic responses to a particular stress, treatment, or disease state. Further, isolation according to the present invention can facilitate understanding of what proteins are expressed in or by a cell at a given stage in development or in response to any natural or synthetic condition. These related areas of research stand to benefit from application of the present isolation methods.

### EXAMPLE IV

### Efficacy of Isolation at Different Stages of Glomeruli Maturation

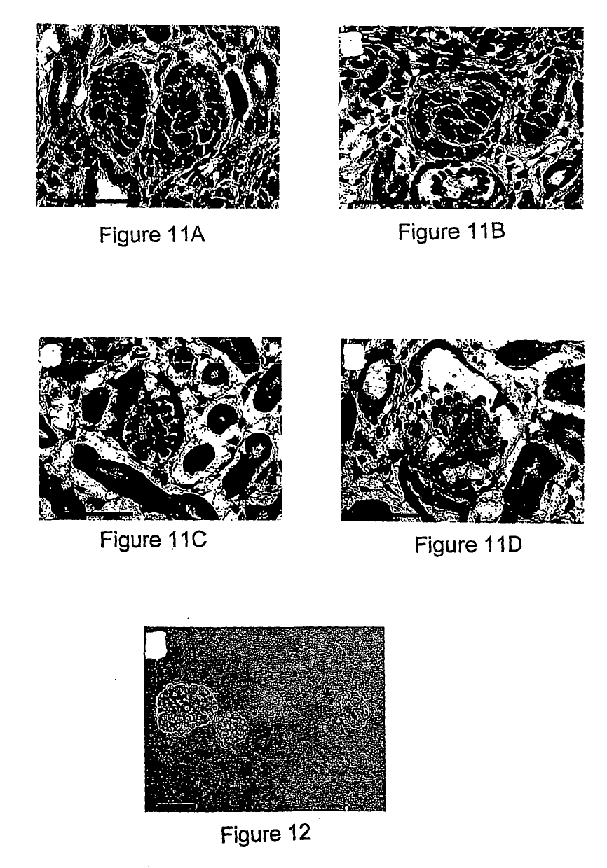

Because glomeruli continue to form and differentiate for up to 2 weeks postpartum¹², newborn mice were used to study whether glomeruli could be collected at different stages of maturation using the novel isolation technique described in Example I. Perfused magnetic beads were distributed in the S-shaped, cup-shaped, and maturing capillary loop stages, but not in the early vesicle stages (analysis from a mouse perfused at post-natal day 2, FIGS. 11A-11D). Developing glomeruli containing magnetic beads were easily isolated using a magnet, contamination of the sample with other renal tissues was low (FIG. 12). Structures reminiscent to the S-shaped, cup-shaped and maturing stages were readily visible and individually collectable using a microscope.

The number of post-natalday 2 mice glomerular structures isolated per mouse was 3560±879 (n=5). RNA isolation revealed that the newborn pup glomeruli contained 30-50 times more RNA per cell than the adult glomerulus cells (data not shown). The demonstrated effectiveness of the isolation method throughout developmental stages makes it a useful research tool.

### EXAMPLE V

### Isolation of Islets of Lanzerhans

Adult mice were anesthetized and perfised with magnetic beads according to Example I. Following perfusion of 2.8 μm through the thoracic aorta, collagenase was perfused through the pancreatic main duct for 5 minutes. The pancreas was removed and minced into to 1 mm³ pieces. The minced pancreas was again subjected to collagenase digestion at 37° C. for 30 minutes. Less time would be required for an immature pancreas. Filtering of the digested pancreatic cells could optionally be performed prior to isolation of the islets. Islets containing magnetic beads were gathered by a MPC and washed at least three times with HBSS at 4° C.

### EXAMPLE VI

### Morhology of Isolated Islets

Histological examination of islets of Langerhans isolated according to Example V revealed magnetic bead distribution throughout the islets (data not shown). The isolation method yielded cells of interest in a relatively pure state and in good condition for observation and further testing. For example, these islet cells could be studied for insulin production parameters and other genetic or proteonomic studies of interest. This would be conducted following the isolation step, by using known methods to isolate or remove the genetic material or the proteins from the isolated cells. These extracts or isolates could be used for further research or analysis. Further discussion of protein isolation can be found in, inter alia, U.S. Pat. No. 5,922,868.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof.

### REFERENCES

1. Lander E. S., et al., Initial Sequencing and Analysis of the Human Genome, Nature 2001; 409:860-921.
2. Venter J. C., et al., The Sequence ofthe Human Genome, Science 2001; 291:1304-1351.
3. Kashtan C. E., et al., Advances in Renal Development, Curr Opin Nephrol Hypertens 2000; 9:247-251.
4. Kuure S., et al., Kidney Morphogenesis: Cellular and Molecular Regulation, Mech Dev 2000; 92:31-45.
5. Schedl A., et al., Cross-talk in Kidney Development, Cuir Opin Genet Dev 2000; 10:543-549.
6. Kashtan C. E., Glomerular Disease, Semin Nephrol 1999; 19:353-363.
7. Lindahl P., et al., Paracrine PDGF-B/PDGF-R beta Signaling Controls Mesangial Cell Development in Kidney Glomeruli, Development 1998; 125:3313-3322.
8. Friedman P. L., et al., Enhanced Visualization of Peripheral Nerve and Sensory Receptors in the Scanning Electron Microscope Using Cryofracture and Osmium-Thiocarbohydrazide-Osmium Impregnation, J Neurocytol 1981; 10:111-131.
9. Braet F., et al., Drying Cells for SEM, AFM and TEM by Hexamethyldisilazane: a Study on Hepatic Endothelial Cells, J Microsc 1997; 186:84-87.
10. Scheidl, J. S., et al., RNA Expression Profiling of Laser Microbeam Microdissected Cells from Slender Embryonic Structure, Am J Pathol. In press.
11. Bonvalet J. P., et al., Number of Glomeruli in Normal and Hypertrophied Kidneys of Mice and Guinea-Pigs. J Physiol 1977; 269:627-41.
12. Reeves W. H., et al., Assembly of the Glomerular Filtration Surface: Differentiation of Anionic Sites in Glomerular Capillaries of Newborn Rat Kidney, J Cell Biol 1980; 85:735-753.
13. Liu A., et al., Neutralizing TGF-betal Antibody Infusion in Neonatal Rat Delays in vivo Glomerular Capillary Formation 1, Kidney Int 1999; 56:1334-1348.
14. Kitamoto Y., et aL, Vascular Endothelial Ggrowth Factor is an Essential Molecule for Mouse Kidney Development: Glomerulogenesis and Nephrogenesis, J Clin Invest 1997; 99:2351-2357.
15. Jeffrey I. K., et al., Isolation and Characterization of Rat Glomerular Epithelial Cells in vivo. Kidney Int 1978; 14:21-30.
16. Greg D., et al., Analysis of Renal Fibrosis in a Rabbit Model of Crescentic Nephritis, J Clin Invest, 1988; 82:998-1006.
17. Baelde J. J., et al., Optimal Method for RNA Extraction from Mouse Glomeruli, Nephrol Dial Transplant 1994; 9:304-308.
18. Kemshead J. T., et al., Magnetic Separation Techniques: Ttheir Application to Medicine, Mol Cell Biochem, 1985; 67:11-18.
19. Kestila M., et al., Positionally Cloned Gene for a Novel Glomerular Protein—Neplrin—is Mutated in Congenital Nephrotic Syndrome, Mol Cell 1998; 1:575-582.
20. Yuan H. T., et al., Expression of Angiopoietin-1, Angippoietin-2, and the Tie-2 Receptor Tyrosine Kinase During Mouse Kidney Maturation, J Am Soc Nephrol 1999; 10:1722-1736.
21. Okubo K., et al., Large Scale cDNA Sequencing for Analysis of Quantitative and Qualitative Aspects of Gene Expression, Nat Genet 1992; 2:173-179.
22. Velculescu V. E., et al., Serial Analysis of Gene Expression, Science 1995; 270:484-487.
23. Schena M., et al., Quantitative Monitoring of Gene Expression Patterns. with a Complementary DNA Microarray, Science 1995; 270:467-70.
24. Jonsson A. P., Mass Spectrometry for Protein and Peptide Characterisation, Cell Mol Life Sci 2001; 58:868-884.

1. A method of isolating target cells, comprising:
perfusing an organism with a solution containing magnetic beads;
removing a selected tissue or region containing target cells from said organism;
digesting said selected tissue or region to separate target cells from associated cell types; and
magnetically isolating target cells from said digested selected tissue or region,
wherein the diameter of said magnetic beads is approximately equivalent to the capillary diameter of the region comprising said target cells.

2. A method of isolating target capillaries, comprising:
perfusing an organism with a solution containing magnetic beads;
removing a selected tissue or region containing target capillaries from said organism;
digesting said selected tissue or region to separate target capillaries from associated cell types; and
magnetically isolating target capillaries from said digested selected tissue or region,
wherein the diameter of said magnetic beads is approximately equivalent to the diameter of the target capillaries.

3. A method of isolating a microorgan, comprising:
perfuising an organism with a solution containing magnetic beads;
removing a selected tissue or region containing the microorgan from said organism;
digesting said selected tissue or region to separate the microorgan from associated cell types; and
magnetically isolating the microorgan from said digested selected tissue or region,
wherein the diameter of said magnetic beads is approximately equivalent to the capillary diameter of the microorgan.

4. A method of isolating a microorgan, comprising:
perfusing an organism with a solution containing magnetic beads;
perfusing an organism with a digestion solution;
removing a selected digested tissue or region containing the microorgan from said organism; and
magnetically isolating the microorgan from said digested selected tissue or region,
wherein the diameter of said magnetic beads is approximately equivalent to the capillary diameter of the microorgan.

5. A method according to claim 1, wherein said digesting is performed using collagenase.

6. A method according to claim 4, wherein said digestion solution is collagenase.

7. A method according to claim 1, further comprising:
filtering said digested selected tissue or region prior to said magnetic isolation step.

8. A method according to claim 7, wherein said filtering is performed using a 100 μm filter.

9. A method according to claim 1, wherein the target cell is selected from the group consisting of: pericytes, vascular smooth muscle cells and astrocytes.

10. A method according to claim 2, wherein the target capillary is selected from the group consisting of dermal capillaries, retinal capillaries, brain capillaries, perineural plexus capillaries, skeletal muscle capillaries, tumor capillaries, heart capillaries, fat capillaries, liver tissue capillaries, intestinal capillaries and capillaries from inflamed tissues.

11. A method according to claim 3, wherein the microorgan is selected from the group consisting of kidney glomeruli, islets of Langerhans and endocrine glands.

12. A method according to claim 4, wherein the microorgan is the islets of Langerhans.

13. A method according to claim 1, wherein the organism is a mammal.

14. A method according to claim 1, wherein the organism is selected from the group consisting of mice, rats, rabbits, guinea pigs, cats, dogs, pigs, cows, monkeys and humans.

15. A method according to claim 1, wherein the organism is at a stage in development selected from the group consisting of embryonic, neonatal, juvenile, and adult.

16. A target cell obtained according to the method of claim 1.

17. A capillary obtained according to the method of claim 2.

18. A microorgan obtained according to the method of claim 3.

19. A microorgan obtained according to the method of claim 4.

20. A method of obtaining genetic material, comprising:
obtaining at least one target cell according to claim 1; and
removing or isolating the genetic material from said at least one target cell.

21. A method of obtaining genetic material, comprising:
obtaining at least one capillary according to claim 2; and
removing or isolating the genetic material from said at least one capillary.

22. A method of obtaining genetic material, comprising:
obtaining at least one microorgan according to claim 3; and
removing or isolating the genetic material from said at least one microorgan.

23. A method of obtaining genetic material, comprising:
obtaining at least one microorgan according to claim 4; and
removing or isolating the genetic material from said at least one microorgan.

24. A method of obtaining proteins, comprising:
obtaining at least one target cell according to claim 1; and
removing or isolating the proteins from said at least one target cell.

25. A method of obtaining proteins, comprising:
obtaining at least one capillary according to claim 2; and
removing or isolating the proteins from said at least one capillary.

26. A method of obtaining proteins, comprising:
obtaining at least one microorgan according to claim 3; and
removing or isolating the proteins from said at least one microorgan.

27. A method of obtaining proteins, comprising:
obtaining at least one microorgan according to claim 4; and
removing or isolating the proteins from said at least one microorgan.

28. A method of obtaining tissue for transplantation, comprising:
obtaining at least one microorgan according to claim 3.

29. A method of obtaining tissue for transplantation, comprising:
obtaining at least one microorgan according to claim 4.
